(19) United States
(12) Patent Application Publication
(10) Pub. No.: US 2024/0157095 A1
(43) Pub. Date: May 16, 2024

(54) NEEDLE AND GUIDE WIRE INSERTION SYSTEM

(71) Applicant: Ann & Robert H. Lurie Children's Hosp. of Chicago, Chicago, IL (US)

(72) Inventors: Paul Tannous, Chicago, IL (US); Alan William Nugent, Dallas, TX (US)

(21) Appl. No.: 18/282,688

(22) PCT Filed: Mar. 22, 2022
(86) PCT No.: PCT/US22/71249

§ 371 Date: Sep. 18, 2023

Related U.S. Application Data
(60) Provisional application No. 63/164,825, filed on Mar. 23, 2021.

(51) Int. Cl. A61M 25/09; A61M 25/06

(52) U.S. Cl. CPC A61M 25/09041; A61M 25/065; A61M 2205/3327; A61M 2205/3331; A61M 2207/10

(57) ABSTRACT

An insertion system includes a main body and a side port mounted at an angle relative to the main body. The side port is sized to receive a guide wire. The insertion system also includes one or more wire guides mounted to the main body and aligned with the side port. The insertion system further includes a handle mounted to the main body.

### CROSS-REFERENCE TO RELATED APPLICATION

The present application claims the priority benefit of U.S. Provisional Patent App. No. 63/164,825 filed on Mar. 23, 2021, the entire disclosure of which is incorporated by reference herein.

### BACKGROUND

The modified Seldinger technique refers to a medical technique in which a guiding sheath is slid over a needle that is inserted into a patient (e.g., into a vessel lumen of the patient). Upon removal of the needle, the guiding sheath is used to guide a wire, catheter, or other device into the patient at the point of interest. The modified Seldinger technique is currently the mainstay method of vascular and other luminal access in interventional cardiology, interventional radiology, and many other fields utilizing percutaneous access. The adoption of ultrasound guidance in conjunction with the modified Seldinger technique has significantly improved the safety profile of the technique by allowing the operator to directly image the needle entering the intended luminal space.

### SUMMARY

An illustrative insertion system includes a main body and a side port mounted at an angle relative to the main body and sized to receive a guide wire. The insertion system also includes one or more wire guides mounted to the main body and aligned with the side port, and a handle mounted to the main body. The main body includes a central lumen, and the side port connects to the central lumen. The insertion system also includes a needle mount mounted to a proximal end of the main body, where the needle mount secures a needle to the main body. In an illustrative embodiment, the needle mount is a male Luer lock.

The insertion system can also include a recessed gutter in the main body, where the recessed gutter is aligned with the side port and the one or more wire guides, and where the recessed gutter receives at least a portion of the guide wire. In an illustrative embodiment, the side port is mounted to a top side of the main body and the handle is mounted to a bottom side of the main body. In another illustrative embodiment, the angle at which the side port is mounted to the main body is between 0 degrees and 90 degrees. In another embodiment, the insertion system includes one or more rollers mounted to a top surface of the main body, where the one or more rollers assist with smooth advancement of the guide wire. In one embodiment, the one or more wire guides comprise L-shaped wire guides. In another embodiment, the one or more wire guides include openings configured to receive the guide wire and guide the guide wire to the side port.

The system can also include an occlusive diaphragm mounted within the side port, and a device mount at a distal end of the main body. The device mount is configured to attach to a pressure transducer to determine a pressure within the main body of the insertion system. The system can also include a guide wire and a needle, where the guide wire is inserted through the side port and into a lumen of the main body, and where the needle is mounted to a proximal end of the main body. In another embodiment, there can be an indicator on the guide wire, where the indicator is positioned such that the indicator is at a distal end of the side port when a proximal end of the guide wire is positioned at a proximal end of the main body. In another embodiment, the system can include a cheater attachment mounted to the side port to facilitate insertion of the guide wire.

An illustrative method of forming an insertion system includes forming a main body that includes a central lumen and forming a side port that connects to the main body at an angle, where the side port is sized to receive a guide wire. The method also includes forming one or more wire guides that connect to a top side of the main body and that are aligned with the side port. The method further includes forming a handle that connects to a bottom side of the main body. In one embodiment, the method includes forming a recessed gutter on the top side of the main body such that the recessed gutter is aligned with the side port and the one or more wire guides. In another embodiment, the one or more wire guides are formed as L-shaped wire guides. The method can also include mounting an occlusive diaphragm mounted over a distal end of the side port. The method can also include mounting a needle mount to a proximal end of the main body, where the needle mount is configured to receive a needle such that a lumen of the needle aligns with the central lumen of the main body. In one embodiment, the method includes forming a guide wire to be inserted into the insertion system, where forming the guide wire includes forming an indicator on the guide wire, where the indicator is positioned such that the indicator is at a distal end of the side port when a proximal end of the guide wire is positioned at a proximal end of the main body.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1A is a side view of an insertion system in accordance with an illustrative embodiment.

FIG. 1B is a top view of the insertion system in accordance with an illustrative embodiment.

FIG. 1C is an end view of the insertion system in accordance with an illustrative embodiment.

FIG. 1D depicts a roller mounted to the main body of the insertion system in accordance with an illustrative embodiment.

FIG. 2 shows a variety of different needles that can be mounted to the needle mount (e.g., male Luer lock) in accordance with an illustrative embodiment.

FIG. 3 depicts a guide wire fed into the insertion system in accordance with an illustrative embodiment.

FIG. 4A depicts a prototype of an insertion system being held by a user in accordance with an illustrative embodiment.

FIG. 4B depicts the prototype of the insertion system with an attached needle and an inserted guide wire in accordance with an illustrative embodiment.

FIG. 5 depicts a cheater attachment in accordance with an illustrative embodiment.

### DETAILED DESCRIPTION

The modified Seldinger technique (MST) is an essential procedure performed over a million times a year. The MST is ubiquitously used for procedures involving percutaneous endoluminal access such as vascular access, pleurocentesis, pericardiocentesis, angiography, chest drains, insertion of pacemaker leads, implantable cardioverter-defibrillators, insertion of percutaneous endoscopic gastrostomy tubes, etc. Ultrasound guidance is often used with MST to confirm needle position within the intended location. However, using traditional MST techniques, the single operator is required to terminate live imaging so that the access needle can be stabilized with one hand, while the other hand advances the guide wire. The advancement of the guide wire is therefore done blindly due to the termination of the live imaging. It is common that access is lost during this transition, with failure to advance the wire, sometimes causing wire injury and/or wire malposition with entry to an undesired location, loss of position, vessel perforation, wall injury, etc. The time that it takes to place the wire in the needle hub can also result in blood loss, particularly in arterial access procedures, and this blood loss can be significant in neonates and infants. Further blood loss, vascular trauma, and hematoma formation can also occur when the wire placement fails for any reason.

Described herein is a needle and guide wire insertion system, which can be referred to as the PXTSidearm. The proposed system is designed to work with any needle and wire combination, and allows a single operator to position and advance a needle and guide wire while using live imaging during the entire insertion procedure. Thus, the proposed system allows the operator to perform the MST while continuously observing needle position and wire advancement, thus increasing procedural safety and reducing the degree of blood loss as compared to traditional systems and techniques.

More specifically, the proposed system integrates a wire loading and delivery system into an access needle. This allows the operator to use a single hand to hold an imaging probe (e.g., an ultrasound probe) for real time imaging, and the other hand to perform the percutaneous stick. When the needle is seen (with the imaging system) entering the desired location, the device allows for wire advancement via the needle with ongoing continuous visualization of needle location. The device also allows for wire delivery out of the needle into the desired location. The proposed system therefore enables a single operator to achieve access to any bodily location with a percutaneous needle followed by wire delivery with continuous imaging. In an illustrative embodiment, the imaging used is ultrasound. In alternative embodiments, any other type of live imaging may be used.

In an illustrative embodiment, the proposed system is compatible with all commercially available access needles and guide wires. In another illustrative embodiment, the proposed system can be a single use device that is disposed after use. In such an embodiment, the device can be made from plastic. Alternatively, the proposed system can be implemented as a multi-use device that is designed to be cleaned and sterilized in between uses. In such a multi-use embodiment, the device can be made from a metal (e.g., stainless steel) and/or a plastic.

In use, a guide wire is loaded into the insertion system, which is attached to the needle of choice. Percutaneous endoluminal access is then established under imaging guidance, such as ultrasound, etc. With the wire in a loaded position, a central lumen of the insertion system remains clear so that the operator can still observe fluid flash in the hub of the needle. Without making any adjustments, the operator is then able to use his/her index finger to advance the guide wire under continuous ultrasound guidance. The proposed insertion system and its use are described in more detail below with reference to the figures.

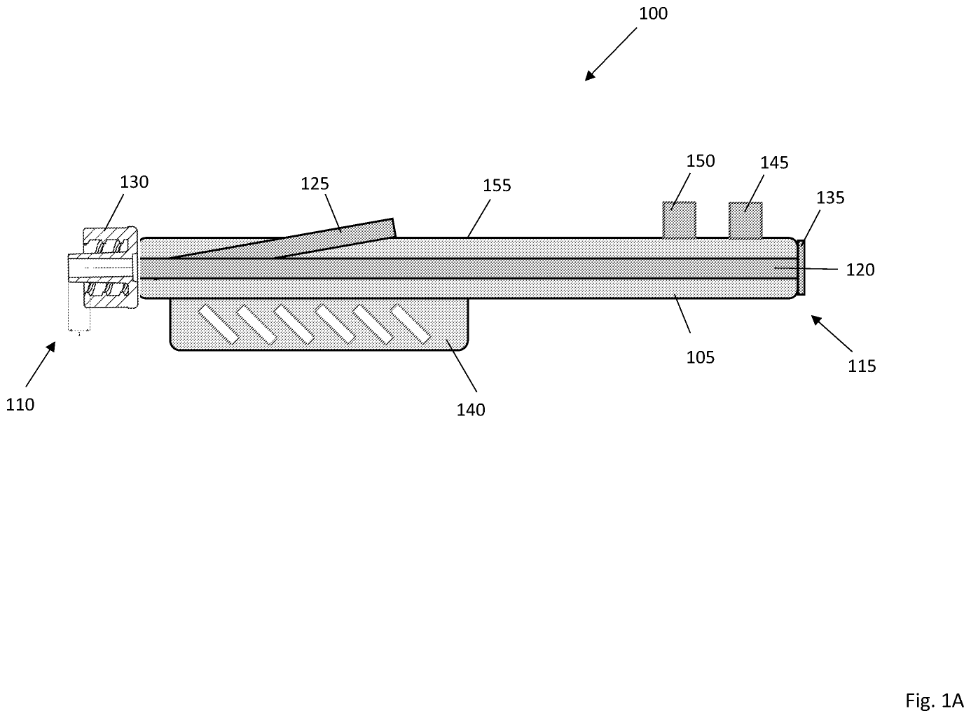

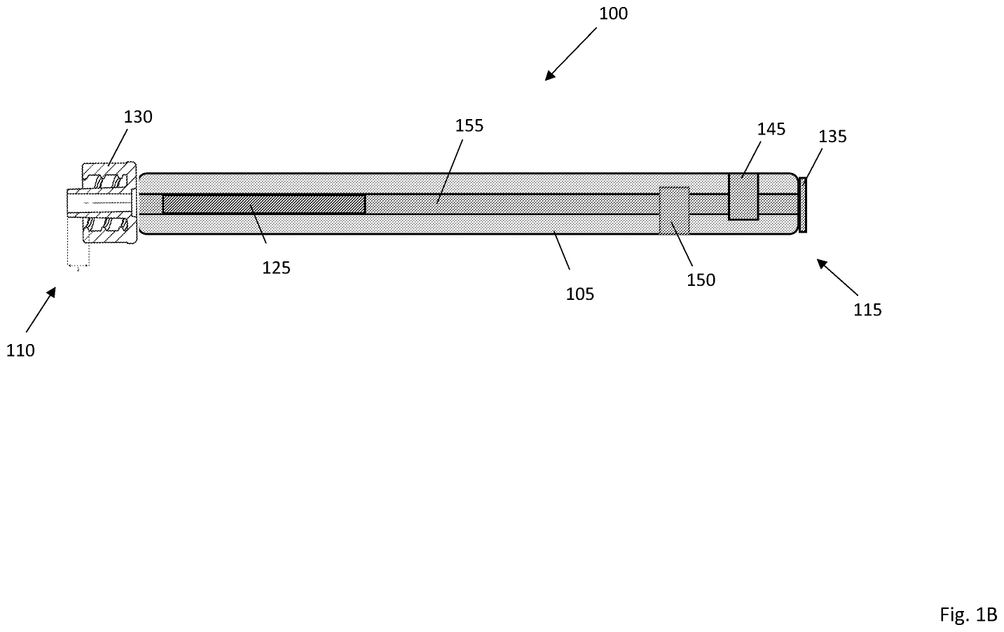

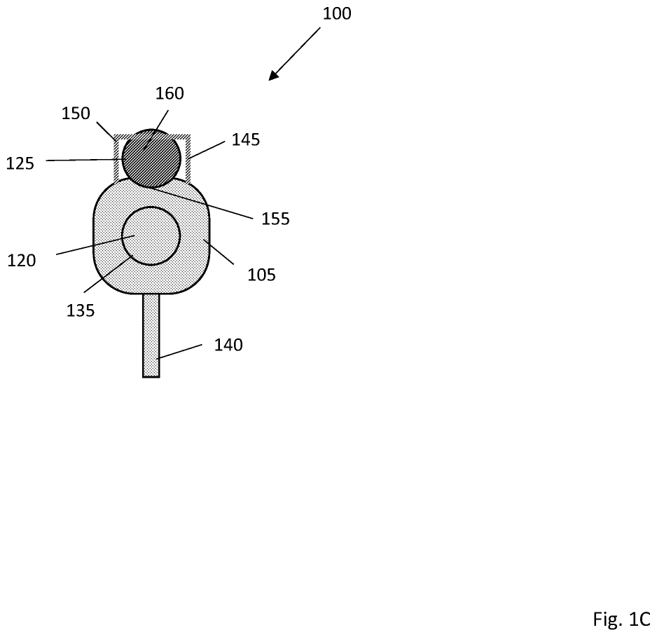

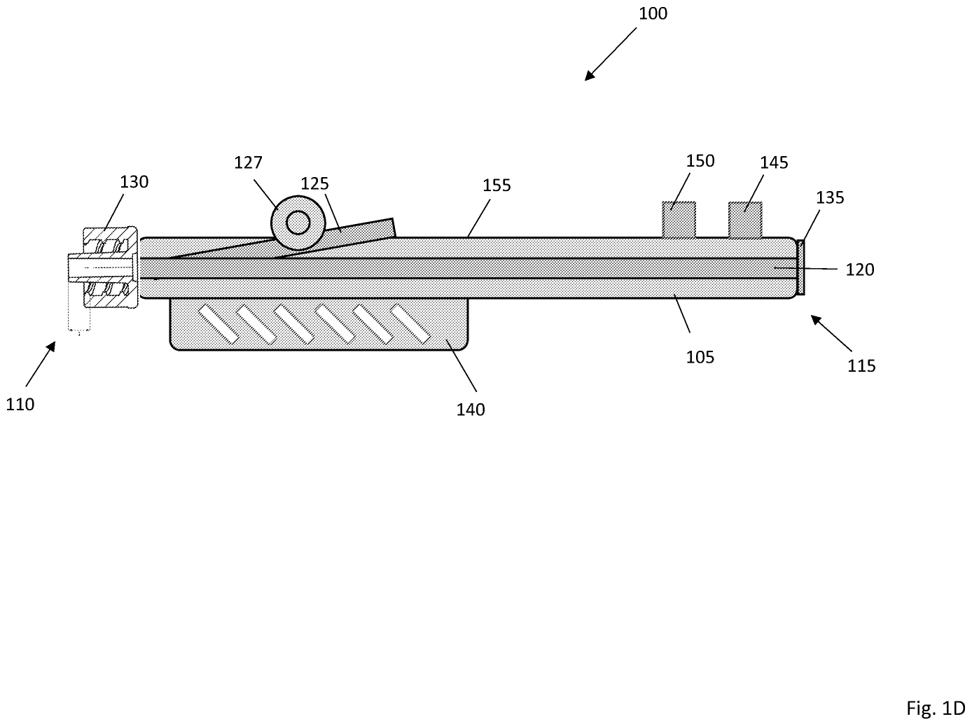

FIG. 1A is a side view of an insertion system 100 in accordance with an illustrative embodiment. FIG. 1B is a top view of the insertion system 100 in accordance with an illustrative embodiment. FIG. 1C is an end view of the insertion system 100 in accordance with an illustrative embodiment. The insertion system 100 includes a main body 105 having a proximal end 110 that is closest to the patient when in use and a distal end 115 that is farthest from the patient when the system is in use. A central lumen 120 runs through a length of the main body 105 of the insertion system 100. In one embodiment, the central lumen can have a diameter of 0.05 inches. Alternatively, a different diameter may be used such as 0.04 inches, 0.06 includes, 0.07 inches, etc. Connected to the central lumen is a side port (or y-port) 125 that provides wire entry access to the central lumen 120. In one embodiment, the side port 125 can have a diameter of 0.04 inches. Alternatively, a different diameter may be used, such as 0.03 inches, 0.05 inches, etc. The side port 125 is mounted at an angle relative to the main body 105 and designed to receive the wire. In an illustrative embodiment, the angle at which the side port 125 is mounted to the main body 105 is approximately 30 degrees, as shown in FIG. 1A. Alternatively, other angles between 0 degrees and 90 degrees may be used, depending on the implementation.

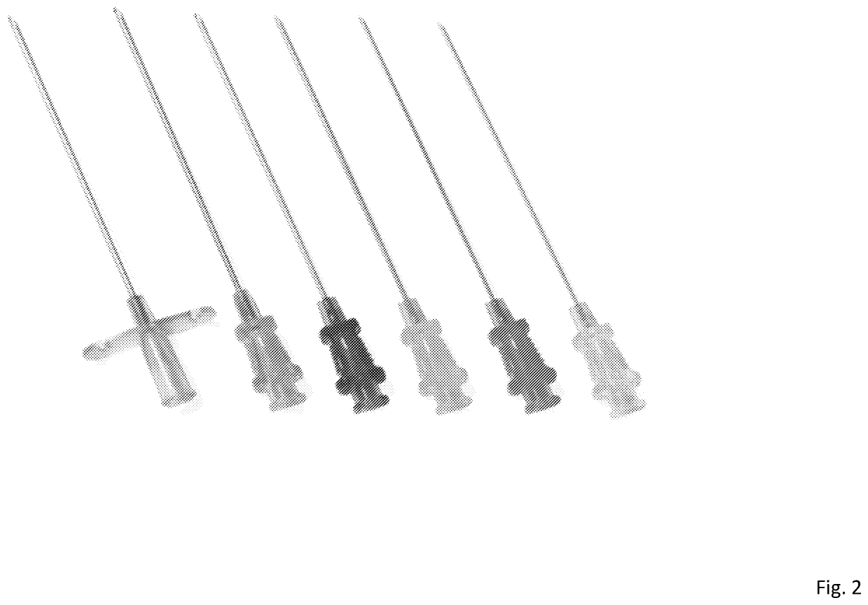

In an illustrative embodiment, a needle mount 130 is mounted to the proximal end 110 of the main body 105 of the insertion system 100. The needle mount 130 is configured to securely receive a needle, and is aligned with the main body 105 such that the central lumen 120 of the main body 105 aligns with a lumen of the needle mount 130. In an illustrative embodiment, the needle mount 130 is a male Luer lock that is designed to be compatible with any commercially available needle in the medical industry. In alternative embodiments, a different type of needle mount may be used, such as threads. FIG. 2 shows a variety of different needles that can be mounted to the needle mount 130 (e.g., male Luer lock) in accordance with an illustrative embodiment. As shown, the insertion system is designed to receive a variety of needles of different diameters and lengths, depending on the needs of the patient. Additionally, the insertion system is compatible with needles other than those depicted in FIG. 2.

Referring again to FIGS. 1A-1C, the distal end 115 of the main body 105 of the insertion system 100 includes a device mount 135. The device mount 135 is used to attach the insertion system 100 to a flushing system so that the unit can be flushed and/or to a pressure transducer. In an illustrative embodiment, the device mount is a female Luer lock. Alternatively, a device mount other than a female Luer lock, such as threads, may be used at the distal end 115 of the main body 105. A handle 140 is also mounted to the main body 105 such that the user is able to hold and manipulate the insertion system 100 with one hand. As shown, the handle 140 is mounted to a bottom portion of the main body 105 toward its proximal end 110. In alternative embodiments, the handle 140 may be mounted elsewhere on the main body 105, such as toward the distal end 115 thereof. Additionally, although the handle 140 is depicted as rectangular in shape with slanted openings to provide a grip for the user, it is to be understood that other shapes and/or configurations of the handle 140 may be used. For example, in one embodiment, the handle 140 can include one or more circular openings mounted to the main body 105, where the one or more circular openings are designed to receive one or more fingers of the user. Other configurations of the handle 140 may also be used in alternative embodiments.

A first wire guide 145 and a second wire guide 150 are mounted to a top surface of the main body 105 of the insertion system 100. In the depicted embodiment, the wire guides are L-shaped. The L-shaped wire guides can be used to feed J-tip and other guide wires into and through the insertion system. In alternative embodiments, the first wire guide 145 and the second wire guide 150 can have a different shape such as a circular opening or a tube with a circular opening, a U-shape, a V-shape, a J-shape, etc. The wire guides are used to help guide the wire into the side port 125, and to maintain the position of the wire relative to the main body 105 such that the user is able to easily feed the wire into the side port 125 with one or more fingers. As best shown in the top view of FIG. 1B, the first wire guide 145 and the second wire guide 150 are staggered along a top surface of the main body 105. Two wire guides are shown in the depicted embodiment. In alternative implementations, a different number of wire guides may be used, such as one, three, four, five, etc. In an illustrative embodiment, the wire guide(s) are configured such that the device is a top-loading system in which the guide wire can be positioned in the needle first, and then secured via the wire guide(s).

As also shown in FIG. 1B, the first wire guide 145 and the second wire guide 150 are mounted over a recessed gutter 155 that runs along at least a portion of the top of the main body 105. The recessed gutter 155 acts in conjunction with the first wire guide 145 and the second wire guide 150 to help maintain the wire in a desired position during insertion of the wire into and through the insertion system 100. The recessed gutter 155 can have a width of 0.04 inches in one embodiment. Alternatively, a different width may be used such as 0.03 inches, 0.05 inches, etc. As shown, the recessed gutter 155 can be an indentation in the form of a curve, or partial circle. Alternatively, a different shape of the indentation may be used, such as a partial (e.g., three-sided) square or rectangle. In one embodiment, one or more rollers can be mounted within the recessed gutter 155 and used to help the guide wire advance more smoothly over the top surface of the main body 105 (i.e., as opposed to sliding the guide wire directly over the recessed gutter 155). In an alternative embodiment, the recessed gutter 155 may not be used, and one or more v-shaped or flat rollers can be mounted to the top surface of the main body 105 to help the guide wire advance smoothly. FIG. 1D depicts a roller 127 mounted to the main body 105 of the insertion system 100 in accordance with an illustrative embodiment.

In the depicted embodiment, the side port 125 is positioned such that at its distal end, a bottom portion of the side port 125 is flush with a top surface of the main body 105. In an alternative embodiment, the side port 125 can be configured such that, at its distal end, a top portion or a mid-portion of the side port 125 is flush with the top surface of the main body 105. In an illustrative embodiment, the side port 125 is aligned with the recessed gutter 155 to facilitate smooth feeding of a guide wire into the insertion system 100.

In one embodiment, the top of the main body 105 can be angled such that the top forms a smooth surface to provide continuous wire to body contact for advancement of the wire. In an illustrative embodiment, the side port 125 of the insertion system 100 allows the user to create a wire holding position in which the user ensures that there is enough wire to be loaded into the system (e.g., into the side port 125), but not within the needle lumen. This allows for bleed back to confirm the position of the needle.

As shown in FIG. 1C, the insertion system 100 includes an occlusive diaphragm 160 within the side port 125. The occlusive diaphragm allows the insertion system to work with a standard pressure transducer/flush system. Specifically, the pressure transducer/flush system can be mounted to the device mount 135, and the occlusive diaphragm enables an air tight connection at the side port 125, which allows the interior (i.e., lumens) of the insertion system 100 to be pressurized. The occlusive diaphragm 160 and pressure transducer allow the operator to further confirm the position of the needle within the artery based on a pressure waveform, which results in zero blood loss for the patient and further increase the safety profile of this procedure. This feature can be very important in neonates and infants, where even a minimal amount of blood loss can be detrimental to the patient. The pressure waveform can be generated by a computing system in communication with the insertion system. The computing system can include a processor, memory, user interface, transceiver (e.g., a receiver and a transmitter), display, etc. The computing system can connect to the insertion system through a wireless or wired communication connection. Pressure data recorded by the transducer can be provided to the computing system and stored in memory. The processor can use the data to generate a pressure waveform that the user can view on a connected display to determine needle position within an artery or other location. In an alternative embodiment, the occlusive diaphragm 160 may not be included, and the end of the side port 125 can be uncovered.

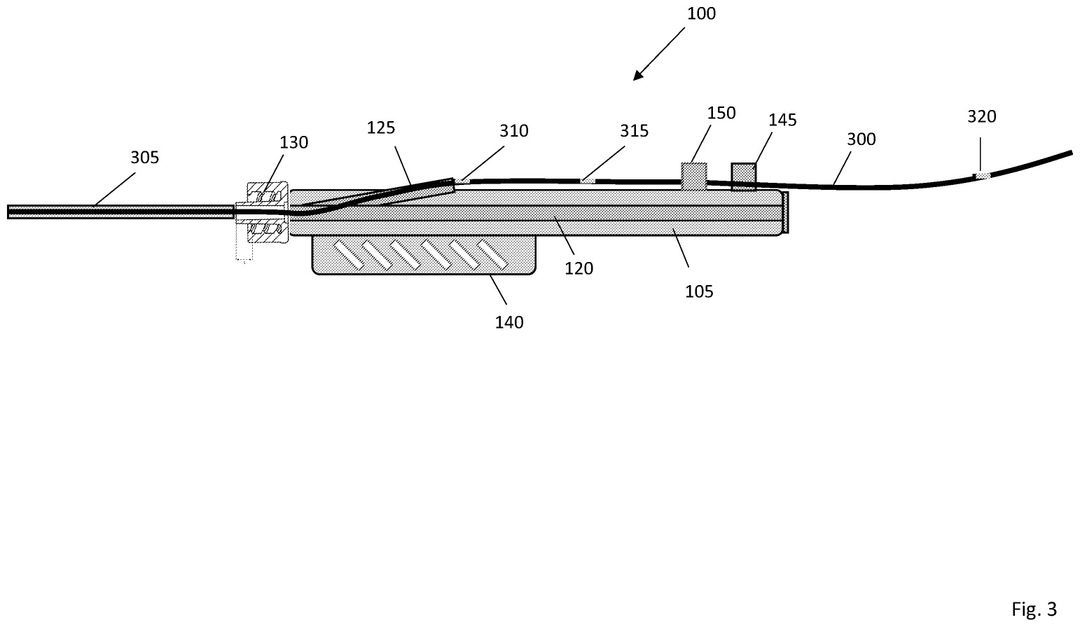

FIG. 3 depicts a guide wire 300 fed into the insertion system 100 in accordance with an illustrative embodiment. As shown, the guide wire 300 runs through the first wire guide 145, through the second wire guide 150, into the side port 125, into the central lumen 120, through the lumen of the male needle mount 130, and into a needle 305 mounted to the needle mount 130. In the embodiment shown, the needle 305 is a 4 centimeter (cm) long needle, although in alternative embodiments needles of different lengths may be used. The guide wire 300 can have various diameters depending on the implementation, such as 0.018 inches, 0.025 inches, 0.035 inches, etc. The guide wire 300 can also have a straight tip, J-tip, or any other type of tip, depending on the procedure.

In the embodiment shown, the guide wire 300 includes markings (e.g., hash marks) or indicators such that the user is aware of the precise location of the end of the wire relative to the needle. Specifically, the guide wire 300 includes a first indicator 310 that, when positioned at the end of the side port 125, informs the user that the guide wire 300 is at the end of the 4 cm long needle 305. The guide wire 300 also includes a second indicator 315 that, when positioned at the end of the side port 125, informs the user that the guide wire 300 is at the end of a 7 cm long needle. The guide wire 300 can similarly include different hash marks (or other indicators) to specify the position of the wire in different length needles such as 3 cm, 5 cm, 6 cm, 8 cm, etc. Also, in alternative embodiments, the indicators on the wire can be aligned with other portions of the insertion system 100 to inform the user of the wire position, such as an edge of one of the wire guides, in between the wire guides, the distal end of the main body 105, etc. As shown, the guide wire 300 also includes a third indicator 320 that indicates when the guide wire 300 is at 20 cm to ensure that the guide wire is beyond the length of the sheath. Another indicator (not depicted) can also be used to indicate when the guide wire 300 is at 30 cm and/or at different distances such as 40 cm, 50 cm, etc.

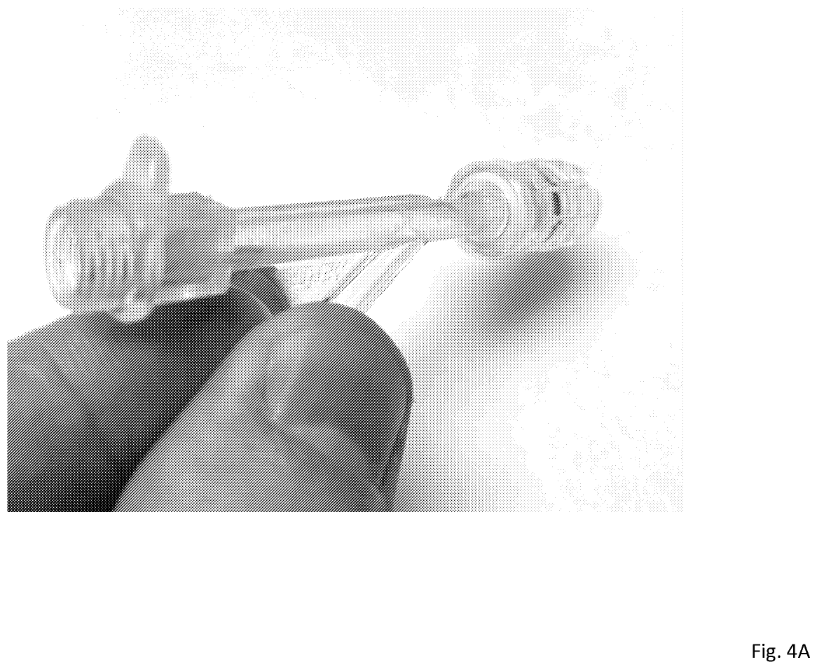

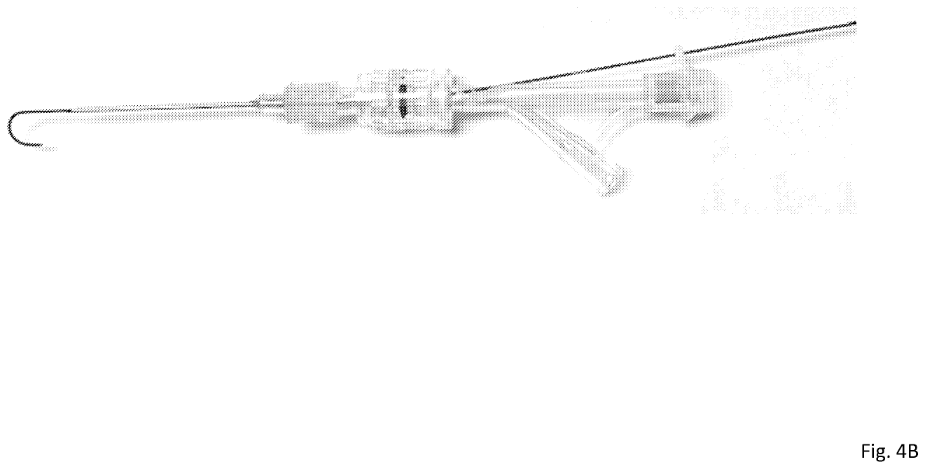

FIG. 4A depicts a prototype of an insertion system being held by a user in accordance with an illustrative embodiment. The user is holding the insertion system by its handle, which is mounted to the bottom of the main body. Also shown are the device mount (e.g., female Luer lock) mounted to the distal end of the main body and the needle mount (e.g., male Luer lock) mounted to the proximal end of the main body. A wire guide in the form of a circular opening and a recessed gutter on the top of the main body can also be seen in FIG. 4A.

FIG. 4B depicts the prototype of the insertion system with an attached needle and an inserted guide wire in accordance with an illustrative embodiment. As shown, the guide wire runs through a wire guide, over/inside a recessed gutter, into and through the side port, into and through the central lumen, into and through the needle mount, and into and through the attached needle such that the guide wire is able to access a desired location within a patient. The same hand that is used to hold the handle of the insertion system can also readily feed the wire into and through the insertion system. For example, a user can hold the insertion system by placing his/her thumb and middle finger on opposite sides of the handle, and use his/her index finger to manipulate the guide wire. As discussed, this allows the other hand of the user to operate an ultrasound imaging system such that the user can accurately visualize both needle and wire placement within the patient during the entire duration of the insertion procedure. In an alternative embodiment, the insertion system can be shortened by shortening the distance between the device mount and the side port and/or by shortening the distance between the needle mount and the side port. In another alternative embodiment, the device can be lengthened such that it is long enough to allow the guide wire to be loaded in the insertion device (i.e., through the side port), but not within the needle lumen. Such an embodiment better facilitates bleed back such that the user can more easily confirm the position of the system.

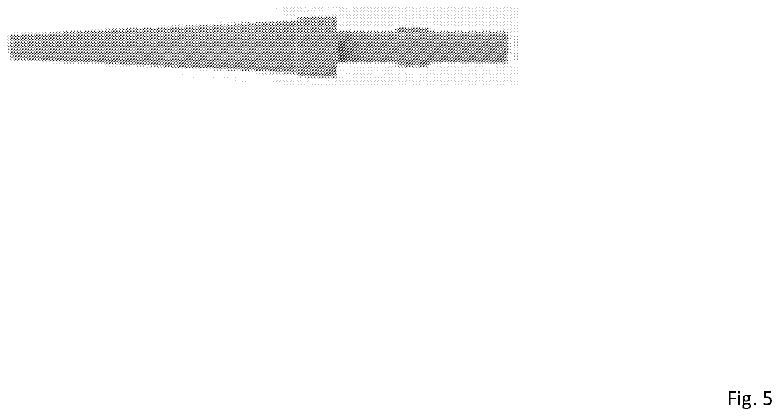

As shown in FIG. 4B, the guide wire has a ‘J’ tip. In alternative embodiments, the tip of the guide wire can be straight or have a different curved shape. In procedures where such a ‘J’ tip guide wire is used, it can potentially be difficult to insert the ‘J’ tip through the side port, into and through the central lumen, into and through the needle mount, and into and through the attached needle. In such a scenario, a cheater attachment can be mounted to the distal end of the side port to help the user guide the wire through the insertion system. In an illustrative embodiment, the side port is sized and shaped such that the cheater attachment can mount to the side port via a friction fit. Alternatively, a threaded connection may be used. FIG. 5 depicts a cheater attachment in accordance with an illustrative embodiment.

Thus, the insertion systems described herein allows for continuous imaging of both the needle and wire during access of endovascular spaces, pleural spaces, pericardial spaces, etc. The proposed system is compatible with any wire and needle system used in the medical profession. In contrast to fixed angle devices that attach to an ultrasound probe, the present insertion system has no restriction on the angle of approach to the patient or on the type of imaging probe used. The proposed insertion system also minimizes blood loss that occurs during wire loading. The insertion system can also have a symmetric or near symmetric design such that a simple mold can be used to manufacture the system. Alternatively, a manufacturing technique other than molding may be used to form the proposed insertion system, such as assembly of components to form the integral device, additive manufacturing, etc. The proposed system is also inexpensive to make, can be disposable, and occupies a small storage space, which makes it ideal for use in a medical setting. Further, as discussed, the proposed insertion system can be used with custom wires that have various diameters, various ends, indicator marks, etc.

The proposed system can be used in any insertion technique to establish endoluminal access, while providing the user with haptic feedback during the insertion process. This endoluminal access can include endovascular access such that arterial and central venous catheters can be positioned in any location within the patient (e.g., femoral artery/vein, radial artery, subclavian artery/vein, transhepatic, etc.). Specifically, the proposed system and techniques can be used to perform central venous catheter placements, arterial line placements, diagnostic cardiac catheterizations, percutaneous coronary interventions, chest tube placement, ultrasound-guided pericardial drain placement, ultrasound-guided gastrostomy placement, pericardiocentisis with drain placement, pleurocentesis with drain placement, paracentesis with drain placement, etc.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. An insertion system comprising:
a main body;
a side port mounted at an angle relative to the main body and sized to receive a guide wire;
one or more wire guides mounted to the main body and aligned with the side port; and
a handle mounted to the main body.

2. The insertion system of claim 1, wherein the main body includes a central lumen, and wherein the side port connects to the central lumen.

3. The insertion system of claim 1, further comprising a needle mount mounted to a proximal end of the main body, wherein the needle mount secures a needle to the main body.

4. The insertion system of claim 3, wherein the needle mount comprises a male Luer lock.

5. The insertion system of claim 1, further comprising a recessed gutter in the main body, wherein the recessed gutter is aligned with the side port and the one or more wire guides, and wherein the recessed gutter receives at least a portion of the guide wire.

6. The insertion system of claim 1, wherein the side port is mounted to a top side of the main body and the handle is mounted to a bottom side of the main body.

7. The insertion system of claim 1, further comprising one or more rollers mounted to a top surface of the main body, wherein the one or more rollers assist with smooth advancement of the guide wire.

8. The insertion system of claim 1, wherein the one or more wire guides comprise L-shaped wire guides.

9. The insertion system of claim 1, wherein the one or more wire guides include openings configured to receive the guide wire and guide the guide wire to the side port.

10. The insertion system of claim 1, further comprising an occlusive diaphragm mounted within the side port.

11. The insertion system of claim 10, further comprising a device mount at a distal end of the main body, wherein the device mount is configured to attach to a pressure transducer to determine a pressure within the main body of the insertion system.

12. The insertion system of claim 1, further comprising a guide wire and a needle, wherein the guide wire is inserted through the side port and into a lumen of the main body, and wherein the needle is mounted to a proximal end of the main body.

13. The insertion system of claim 12, further comprising an indicator on the guide wire, wherein the indicator is positioned such that the indicator is at a distal end of the side port when a proximal end of the guide wire is positioned at a proximal end of the main body.

14. The insertion system of claim 1, further comprising a cheater attachment mounted to the side port to facilitate insertion of the guide wire.

15. A method of forming an insertion system, the method comprising:
forming a main body that includes a central lumen;
forming a side port that connects to the main body at an angle, wherein the side port is sized to receive a guide wire;
forming one or more wire guides that connect to a top side of the main body and that are aligned with the side port; and
forming a handle that connects to a bottom side of the main body.

16. The method of claim 15, further comprising forming a recessed gutter on the top side of the main body such that the recessed gutter is aligned with the side port and the one or more wire guides.

17. The method of claim 15, wherein the one or more wire guides are formed as L-shaped wire guides.

18. The method of claim 15, further comprising mounting an occlusive diaphragm mounted over a distal end of the side port.

19. The method of claim 15, further comprising mounting a needle mount to a proximal end of the main body, wherein the needle mount is configured to receive a needle such that a lumen of the needle aligns with the central lumen of the main body.

20. The method of claim 15, further comprising forming a guide wire to be inserted into the insertion system, wherein forming the guide wire includes forming an indicator on the guide wire, wherein the indicator is positioned such that the indicator is at a distal end of the side port when a proximal end of the guide wire is positioned at a proximal end of the main body.
